(19) United States
(12) Patent Application Publication
(10) Pub. No.: US 2011/0251377 A1
(43) Pub. Date: Oct. 13, 2011

(54) IONIC LIQUID SYSTEMS FOR THE PROCESSING OF BIOMASS, THEIR COMPONENTS AND/OR DERIVATIVES, AND MIXTURES THEREOF

(75) Inventors: Mustafizur Rahman, Tuscaloosa, AL (US); Ning Sun, Tuscaloosa, AL (US); Ying Qin, Tuscaloosa, AL (US); Mirela L. Maxim, Tuscaloosa, AL (US); Robin D. Rogers, Tuscaloosa, AL (US)

(73) Assignee: THE BOARD OF TRUSTEES OF THE UNIVERSITY OF ALABAMA, Tuscaloosa, AL (US)

(21) Appl. No.: 13/129,060

(22) PCT Filed: Nov. 12, 2009
(86) PCT No.: PCT/US09/64105

§ 371 Date: Jun. 15, 2011

Related U.S. Application Data
(60) Provisional application No. 61/113,716, filed on Nov. 12, 2008.

(51) Int. Cl. C08H 7/00; C08B 1/00; C07H 1/06

(52) U.S. Cl. 530/500; 536/123.1; 536/56

(57) ABSTRACT

Disclosed herein are compositions and methods that involve ionic liquids and biomass. In one aspect, the disclosure relates to ionic liquid systems for the processing of biomass, their components and/or derivatives, and mixtures thereof.

### BACKGROUND

With an ever-increasing awareness of global energy consumption and related environmental concerns, demands for “green” industrial processes are growing. An industrial move toward more environmentally benign practices may become inevitable as local and global credits for such practices become more mainstream, and, likewise, as anticipated “carbon taxes” drive markets away from environmentally malignant practices. One way of enhancing industrial environmental stewardship and avoiding these potential financial pitfalls is through the retrieval and use of biorenewable materials for industrial applications. Efficient and “green” biomass processing can, for example, transform relatively cheap, crude natural materials such as trees and crops into materials useful in a number of markets including paper and pulp, pharmaceuticals, and commodity chemicals, to name a few.

In paper and pulp industries, for example, a need exists for improved methods aimed at processing lignocellulosic biomass. Specifically, the fractionation and retrieval of biomass constituents can be a difficult challenge, and a need exists for improved processes directed at the fractionation and extraction of biomass components, particularly since the uses biomass components are so widespread. Cellulose, for example, is used as paper, glucose, and alcohol precursors, while lignin finds use in binders, dispersants, emulsifiers, and recently, in carbon fiber materials. With pulp mill sales reaching $34 billion during 2006 and annual growth rates for this industry projected to be between 2 and 8% in North and South America, it is readily apparent that a more efficient lignocellulosic biomass processing method could lead to increased profits for this industry.

Thus, a need for improved and “green” separation techniques for biomass, and more specifically, improved lignocellulosic biomass separation techniques, exists. This need and other needs are at least partially satisfied by the multiphasic compositions and methods of using such compositions disclosed herein.

### SUMMARY

In accordance with the purposes of the disclosed materials, compounds, compositions, articles, and methods, as embodied and broadly described herein, the disclosed subject matter, in one example, relates to methods comprising the dissolution of lignocellulosic biomass in ILs, and separation of the components by using appropriate solvents and/or by distillation.

Additional advantages of the disclose subject matter will be set forth in part in the description that follows, and in part will be obvious from the description, or can be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a diagram of formulas which represent (a) cellulose, (b) xylan hemicellulose, and (c) lignin.

FIG. 2 is ¹³C NMR spectra of (from top) MCC in C₂mimOAc/DMSO-d6, xylan in DMSO d6, Indulin AT in DMSO d6, and Southern Yellow Pine in C₂mimOAc/DMSO-d6.

FIG. 3 is IR spectra of original (bottom) and recovered C₂mimOAc (top).

FIG. 4 is ¹H NMR spectra of original (top) and recovered (bottom) C₂mimOAc in DMSO d6. Recovered C₂mimOAc has additional peaks from xylan (circled).

FIG. 5 is ¹³C NMR spectra of xylan (top) and recovered C₂mimOAc in DMSO d6 (bottom).

FIG. 6 is IR spectra of Indulin AT (bottom) and regenerated Indulin AT from C₂mimOAc (top).

FIG. 7 is ¹³C NMR spectra of original (bottom) and regenerated (top) Indulin AT in DMSO-d6.

FIG. 8 is IR spectra of xylan regenerated from KOH solution (top), pure xylan (middle), and xylan regenerated from water (bottom).

FIG. 9 is IR spectra of pure xylan (top), xylan regenerated from DMSO, and pure DMSO (bottom).

FIG. 10 is ¹³C NMR of original (bottom) and regenerated (top) xylan in DMSO-d6.

FIG. 11 is IR spectra of a microcrystalline cellulose film (bottom) and the film regenerated from three standards from C₂mimOAc (top).

FIG. 12 is XRD spectra of a microcrystalline cellulose film regenerated from plain microcrystalline cellulose solution in C₂mimOAc (A) and from all standards dissolved in C₂mimOAc after other components have been washed out (B). While intensity in the samples varies, the peaks appear to be the same in both cases.

FIG. 13 is an exemplary scheme for the dissolution of lignocellulosic biomass in an IL and separation of the components.

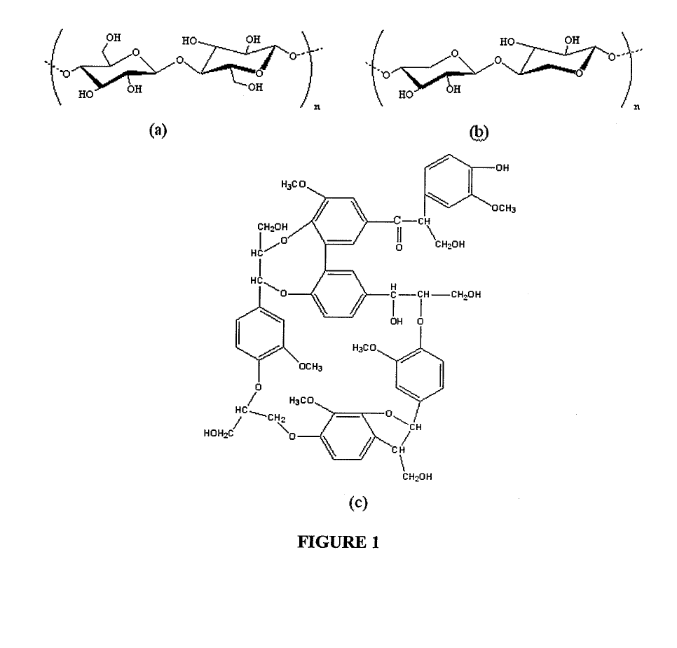

### DETAILED DESCRIPTION

The materials, compounds, compositions, and methods described herein may be understood more readily by reference to the following detailed description of specific aspects of the disclosed subject matter, the Figures, and the Examples included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such component, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. “About” can mean within 5% of the stated value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “2000” is disclosed, then “about 2000” is also disclosed. It is also understood that when a value is disclosed, then “less than or equal to” the value, “greater than or equal to the value” and possible ranges between values are also disclosed, as appropriately understood by the skilled artisan. For example, if the value “2000” is disclosed, then “less than or equal to 2000” as well as “greater than or equal to 2000” is also disclosed. It is also understood that throughout the application data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound comprising 2 parts by weight of component X and 5 parts by weight component Y, X and Y are present at a weight ratio of 2:5, and are present in such ratio regardless of whether additional components are comprised in the composition.

A weight percent (wt. %) of a component, unless specifically stated to the contrary, is based on the total weight of the formulation or composition in which the component is included.

As used herein, the terms “fraction,” “fractioning,” and “fractionation” refer to a process comprising separating a mixture into quantities or components. If a mixture comprises, for example, two components, fractioning or fractionation of the mixture can comprise complete or partial separation of the two components. A “fractionation composition” is a composition that can be used to fraction a mixture.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

“A¹,” “A²,” “A³,” and “A⁴” are used herein as generic symbols to represent various substituents. These symbols can be any substituent, not limited to those disclosed herein, and when they are defined to be certain substituents in one sentence it does not mean that, in another sentence, they cannot be defined as some other substituents.

The term “alkyl” as used herein is a branched or unbranched saturated hydrocarbon group of 1 to 24 carbon atoms, such as methyl (C₁), ethyl (C₂), n-propyl (C₃), isopropyl (C₃), n-butyl (C₄), isobutyl (C₄), t-butyl (C₄), pentyl (C₅), hexyl (C₆), heptyl (C₇), octyl (C₈), nonyl (C₉), decyl (C₁₀), dodecyl (C₁₂), tetradecyl (C₁₄), hexadecyl (C₁₆), octadecyl (C₁₈), eicosyl (C₂₀), tetracosyl (C₂₄), and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below. Abbreviations for various alkyl groups used herein are as follows: Me is methyl (CH₃), Et is ethyl (C₂H₅), Pr is propyl (C₃H₇), Bu is butyl (C₄H₉), etc.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” specifically refers to an alkyl group that is substituted with one or more halides, e.g., fluorine, chlorine, bromine, or iodine. The term “alkoxyalkyl” specifically refers to an alkyl group that is substituted with one or more alkoxy groups, as described below. The term “alkylamino” specifically refers to an alkyl group that is substituted with one or more amino groups, as described below, and the like. When “alkyl” is used in one instance and a specific term such as “alkylalcohol” is used in another, it is not meant to imply that the term “alkyl” does not also refer to specific terms such as “alkylalcohol” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “alkoxy” as used herein is an alkyl group bound through a single, terminal ether linkage.

The term alkoxylalkyl as used herein is an alkyl group that comprises an alkoxy substituent.

The term “alkenyl” or “alkene” or “alkylene” as used herein is a hydrocarbon group of from 2 to 24 carbon atoms with a structural formula comprising at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that comprises any carbon-based aromatic group including, but not limited to, benzene, naphthalene, phenyl, biphenyl, phenoxybenzene, and the like. The term “aryl” also includes “heteroaryl,” which is defined as a group that comprises an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that comprises an aromatic group that does not comprise a heteroatom. The aryl group can be substituted or unsubstituted. The aryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can comprise one or more aryl groups, one or more non-aryl groups, or one or more aryl groups and one or more non-aryl groups.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture.

The general term “polymer” includes homopolymer, copolymer, terpolymer, natural and synthetic polymers, biopolymers, fractionation polymers, etc. unless the context clearly dictates otherwise. When the prefix “poly” is used, reference is made to the product of polymerization of a monomer. Thus, the term “polyalkylene glycol” includes any polymerization product of the alkylene glycol monomer to which reference is made. The specific term “fractionation polymer” is used herein to identify a polymer that separates into its own phase when admixed with an ionic liquid at a given set of parameters, as are described herein for use in the disclosed multiphasic fractionation processes. This term is used as a mere aid to distinguish such polymers from among the various polymer components of biomass (e.g., polysaccharides proteins), which can be also present in the system.

Molecular weights can be expressed in units of molecular mass, i.e., g/mol, or more broadly in units of atomic mass, i.e., Daltons. These two unit expressions can be use interchangeably and, for the purposes of this disclosure, are synonymous. When in reference to a polymer, molecular weights can or cannot be the true molecular weight of the disclosed polymer. Also, disclosed polymer molecular weights can often represent a value advertised by a commercial supplier and/or molecular weights determined through reference of a polymer standard using, for example, liquid chromatography. This disclosure does not intend to be limited by this practice as those skilled in art are aware of these conventions.

Unless otherwise specified, a “molecular weight” of a polymer refers to the relative average chain length of the bulk polymer. In practice, molecular weight can be estimated or characterized in various ways including gel permeation chromatography (GPC) or capillary viscometry. GPC molecular weights are reported as the weight-average molecular weight (Mw) as opposed to the number-average molecular weight (Mn). Capillary viscometry provides estimates of molecular weight as the inherent viscosity determined from a dilute polymer solution using a particular set of concentration, temperature, and solvent conditions.

The term “number average molecular weight” (Mₙ) is defined herein as the mass of all polymer molecules divided by the number of polymer molecules which are present.

The term “weight average molecular weight” (M_{w}) is defined herein as the mass of a sample of a polymer divided by the total number of molecules that are present.

The term “polydispersity” or “polydispersity index” or “PDI” is defined herein as the weight average molecular weight, M_{w}, divided by the number average molecular weight, Mₙ.

The term “processing” is used herein to generally refer to the various treatments that a biomass can undergo, for example, physical treatments such as mixing, fractioning, drying, dying, and chemical treatments such as degradation, delignification, derivatization, functional group transformation (e.g., acetylation and deacetylation), fermentation, and the like.

Also, disclosed herein are materials, compounds, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a composition is disclosed and a number of modifications that can be made to a number of components of the composition are discussed, each and every combination and permutation that are possible are specifically contemplated unless specifically indicated to the contrary. Thus, if a class of components A, B, and C are disclosed as well as a class of components D, E, and F and an example of a composition A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to, compositions and steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

Reference will now be made in detail to specific aspects of the disclosed materials, compounds, compositions, components, devices, articles, and methods, examples of which are illustrated in the following description and examples, and in the figures and their previous and following description.

### Materials and Methods

Disclosed herein are various compositions and methods that involve the use of ionic liquids (ILs) and mixtures of ionic liquids for processing biomass. For example, in one embodiment, disclosed are compositions and methods for fractioning various components in biomass using conventional solvent systems. In a further embodiment, ILs are used to dissolve biomass and processing aids in order to process and transform biomass and components thereof. In a still further embodiment, multiple IL systems comprising a biomass or components thereof are disclosed.

Biomass Dissolution and Component Separation

It can be difficult to separate the components of lignocellulosic biomass without the degradation of one or more of the components. However, biomass components, by themselves, differ in structural properties and hence can be dissolved in different solvents to different extents. During dissolution of lignocellulosic biomass in an IL, partial delignification occurs simultaneously, rendering free components in the IL-solution. Thus, in one aspect, the present invention relates in part to the dissolution of lignocellulosic biomass and later, the selective fractionation, e.g., separation of biomass components using suitable solvents. The separation biomass components can optionally be regenerated.

In one aspect, a method for the dissolution and separation of biomass can be carried out at any appropriate temperature. However, it should be appreciated that the disclosed methods can be carried out at room temperature. In a further aspect, a method can be cost-effective by using cheap and/or recyclable solvents. Furthermore, dissolved components can be easily recovered and the solvents can be recycled. In a further aspect, volatile aromatics present in lignocellulosic biomass (as in scented wood) can also be extracted using this a disclosed method.

In one aspect, a solvent suitable for regenerating biomass components includes, but is not limited to, alcohol, ether, aldehyde, ketone, carboxylic acid and their esters, acetonitrile and other hydrocarbon-based solvents, water, and aqueous solutions of any of these solvents.

The discovery of dissolution of cellulose in IL involved the use of C₄mimCl (Swatloski, R. P.; Spear, S. K.; Holbrey, J. D.; Rogers, R. D. Dissolution of cellulose with ionic liquids. J. Am. Chem. Soc. 2002, 124, 4974-4975.), which is incorporated by reference herein in its entirety for its teaching of cellulose dissolution in IL. Thus, in one aspect, C₄mimCl can be used.

Typically, dissolution of cellulose can require high temperatures to provide energy sufficient to break hydrogen bonds. Therefore, dissolution of biomass components can be carried out at elevated temperatures (about 90° C.), as in studies (Fort, D. A.; Remsing, R. C.; Swatloski, R. P.; Moyna, P.; Moyna, G.; Rogers, R. D. Can ionic liquids dissolve wood? Processing and analysis of lignocellulosic materials with 1-n-butyl-3-methylimidazolium chloride. Green Chem. 2007, 9, 63-69, which is incorporated by reference herein in its entirety for its teaching of ionic liquid dissolution of lignocellulosic materials.). A number of organic solvents, commonly used in both laboratory and commercial scale, can be used as separation solvents. Alternatively, the present methods can be carried out at room temperatures, or temperatures that vary through the course of the method.

It will be apparent that each component of lignocellulosic biomass can show considerable solubility in the exemplary ILs tested (Table 1). However, their solubility can vary when studied with conventional laboratory chemicals and their aqueous solutions (Table 2). From the solubility data, it can be evident how different solvents can be used to extract these components from a homogeneous solution. Specific solvent(s) can be used to dissolve and wash out a particular component, leaving others in the mixture.

In one example, in order to study a disclosed fractionation protocol as if the primary lignocellulosic bonds are already broken, commercially available biopolymer standards were mixed and dissolved in C₂mimOAc to mimic the delignified biomass. MCC is a known cellulose standard and has been studied widely (El Seoud, O. A.; Koschella, A.; Fidale, L. C.; Dorn, S.; Heinze, T. Applications of ionic liquids in carbohydrate chemistry: A window of opportunities. Biomacromol. 2007, 8(9), 2629-2647.). Hemicellulose, on the other hand, is a heteropolymer and is comprised of varying compositions of xylan, glucuronoxylan, arabinoxylan, glucomannan and xyloglucan. Xylan is a major component of hemicellulose and was studied as the hemicellulose standard since it represents typical hemicellulosic properties and is commercially available. Indulin AT, which is a purified form of Kraft pine lignin, was studied as a lignin standard. Since natural lignin has a complex structure and appears in variety of crosslinked forms, Indulin AT is considered as one of the model lignin compounds and has often been studied as a lignin standard (Cateto, C. A.; Barreiro, M. F.; Rodrigues, A. E. Monitoring of lignin-based polyurethane synthesis by FTIR-ATR. Ind. Crops Prod. 2008, 27(2), 168-174; Manangeeswaran, M.; Ramalingam, V. V.; Kumar, K.; Mohan, N. Degradation of indulin, a kraft pine lignin, by Serratia marcescens. J. Environ. Sci. Health, Part B: Pesticides, Food Contaminants, and Agricultural Wastes, 2007, 42(3), 321-327; Willauer, H. D.; Huddleston, J. G.; Li, M.; Rogers, R. D. Investigation of aqueous biphasic systems for the separation of lignins from cellulose in paper pulping process. J. Chromatogr. B: Biomed. Sci. Applic. 2000, 743 (1-2), 127-135, which are all incorporated by reference herein in their entireties for their teachings of lignocellulosic separation techniques.).

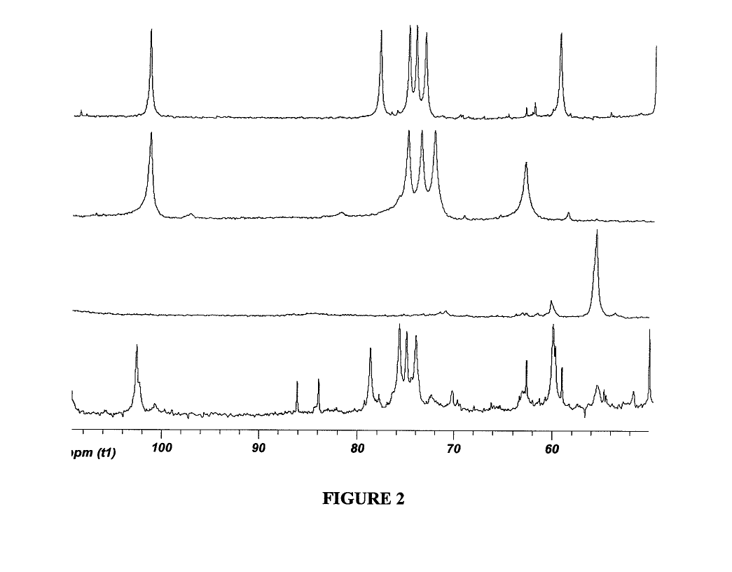

FIG. 2 shows the ¹³C NMR spectra of example individual components, selected from mimic lignocellulosic biomass components (i.e., Indulin AT for lignin, xylan for hemicellulose, and microcrystalline cellulose for cellulose) and Southern Yellow Pine sawdust dissolved in C₂mimOAc. While the peaks arising from individual biopolymers are shown in the top three spectra, contribution of these components in the spectrum of sawdust is evident from the bottom spectrum. Additional peaks in the pine spectra are due to hemicellulose contents (other than xylan) not studied as individual biopolymer standard.

TABLE 1
Solubility (wt %) of standards in ILs at about 90° C.

| IL | MCC | Xylan | Indulin AT |
|---|---|---|---|
| C₄mimCl | ~28 | ~8 | ~14 |
| C₂mimCl | ~35 | ~10 | ~20 |
| C₂mimOAc | ~30 | ~10 | >25 |

TABLE 2
Solubility (wt %) of standards in different regeneration
solvents at about 23° C.

| Solvent | MCC | Xylan | Indulin AT |
|---|---|---|---|
| water | <0.1 | >10 | <0.1 |
| 3:7 acetone-water | <0.1 | <0.1 | <1 |
| 1:1 acetone-water | <0.1 | <0.1 | ~13 |
| 7:3 acetone-water | <0.1 | <0.1 | ~9 |
| Acetone | <0.1 | <0.1 | <0.5 |
| 1:1 ethanol-water | <0.1 | <0.1 | <0.5 |
| 7:3 ethanol-water | <0.1 | <0.1 | ~2 |
| Ethanol | <0.1 | <0.1 | <0.5 |
| Acetonitrile | <0.1 | <0.1 | <0.5 |
| Cyclohexane | <0.1 | <0.1 | <0.5 |
| Dichloroethane | <0.1 | <0.1 | <0.5 |
| Dichloromethane | <0.1 | <0.1 | <0.1 |
| Diethyl ether | <0.1 | <0.1 | <0.1 |
| Hexane | <0.1 | <0.1 | <0.1 |
| Methylisobutylketone | <0.1 | <0.1 | <0.5 |
| 1 wt % KOH solution | <0.2 | >10 | >10 |
| DMSO^{†} | <0.1 | >20 | >20 |

^{†}value reported at about 90° C.

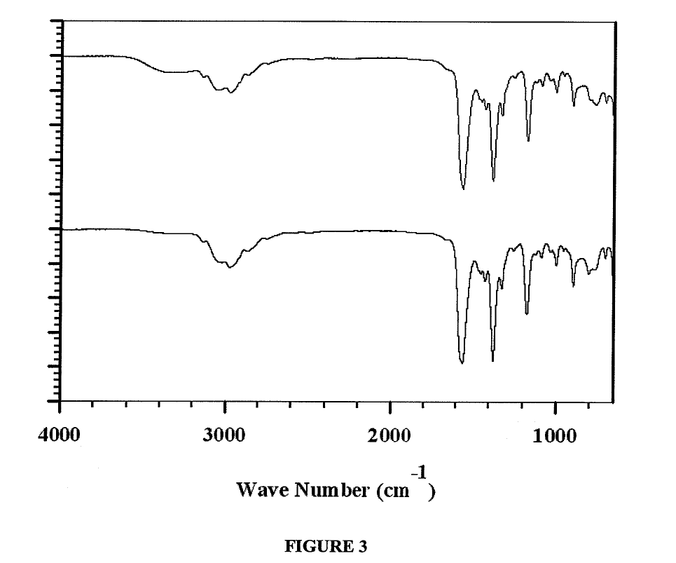

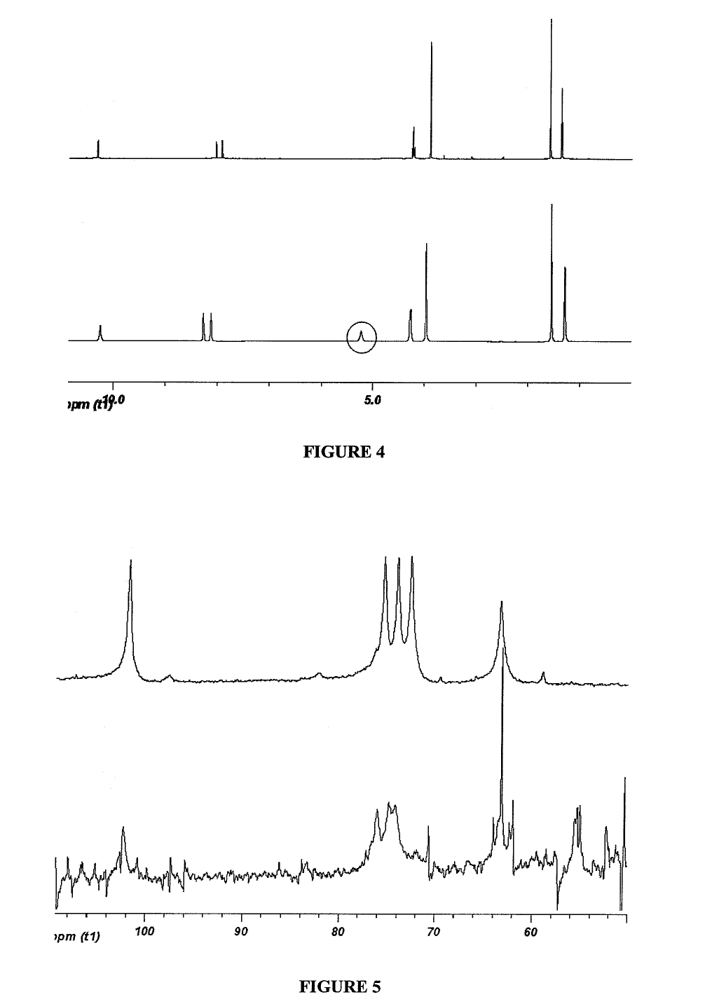

In one aspect, a biomass solution can be dissolved in an ionic liquid. In a further aspect, after the biopolymers are dissolved in an ionic liquid, a film can be cast from this solution, and the film can be processed. Subsequently, the ionic liquid can be washed out with water to obtain an aqueous solution of ionic liquid. To recycle the ionic liquid, the extracted solution can be dried. For example, in one method, approximately 9.94 g of C₂mimOAc, with some dissolved xylan was recovered. IR analysis of recovered C₂mimOAc (FIG. 3) showed that the IL had been recovered without any degradation (and can be recycled later). ¹H NMR (FIG. 4) also confirmed the unaltered IL structure, with extraction of some xylan by water (with IL). ¹³C NMR (FIG. 5) spectra confirmed presence of xylan in the recovered IL.

In one aspect, once the components of biomass are dissolved in an ionic liquid solution, the solution itself can be processed according to disclosed methods.

In a further aspect, an ionic liquid can be allowed to leach out of a film, as an alternative to washing out of solution or out of a film. In one example, C₂mimOAc was allowed to leach out of a film cast from a biomass solution. In this example, similar results to the washing protocol were obtained. In yet a further aspect, a cast film can be washed with hot water for simultaneous extraction of IL and xylan (and/or hemicellulose) for potentially an enhanced ‘extraction’ effect.

In one aspect, provided are methods for extracting lignin from a biomass solution or film. Any appropriate solvent can be used for this step. In one aspect, a suitable solvent for lignin extraction is a water acetone mixture. For example, Indulin AT was washed out of a film by a 1:1 acetone-water solution at room temperature (see Table 2 for solubility analysis). In this example, most of the other dissolved biopolymers remained in the film.

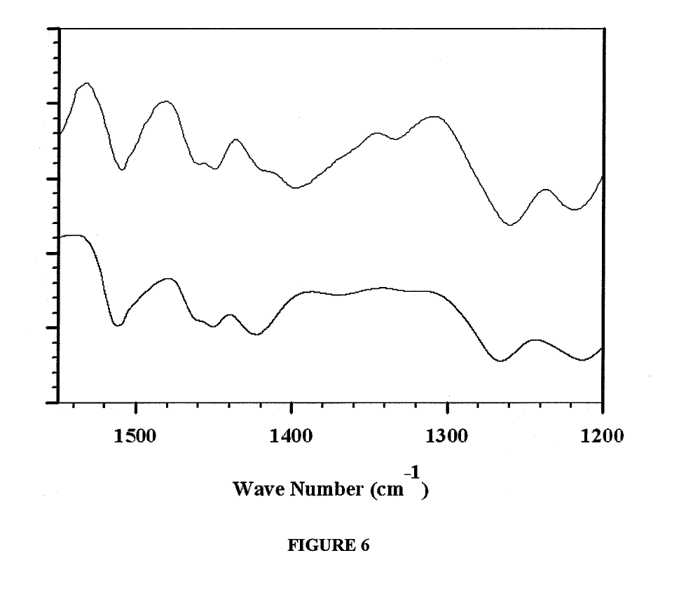

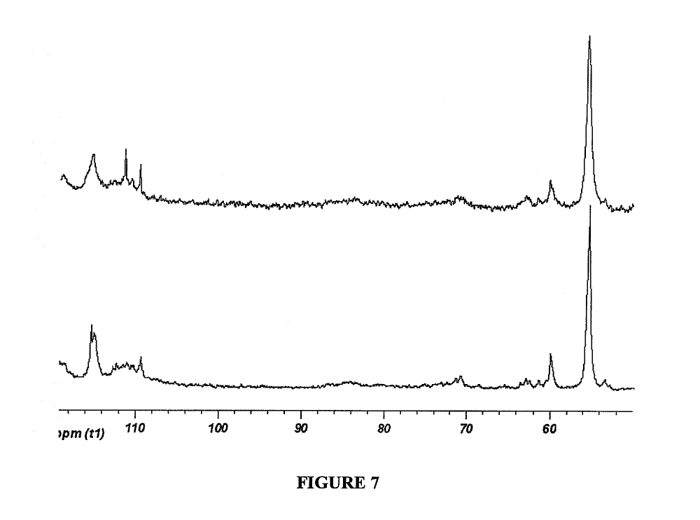

In a further aspect, lignin can be conveniently recovered from an extracted lignin solution by evaporating acetone. As an example, evaporation of acetone from a solution of extracted Indulin AT rendered Indulin AT insoluble in remaining water (Table 2) and the Indulin AT precipitated from the solution. The recovered Indulin AT was dried and then characterized by IR (FIG. 6) and NMR (FIG. 7) analyses. Peak positions of Indulin AT in both cases were found to remain unchanged. 0.22 g of Indulin AT could be recovered using this example method.

In one aspect, provided are methods for removing hemicellulose (e.g., at least one xylan) from a biomass solution or film. Any appropriate solvent can be used to remove at least one xylan from a biomass solution. For example, according to Table 2, there are at least two solvent systems (5 wt % aqueous KOH solution at room temperature and DMSO at about 90° C.). which can be used for separating xylan from cellulose.

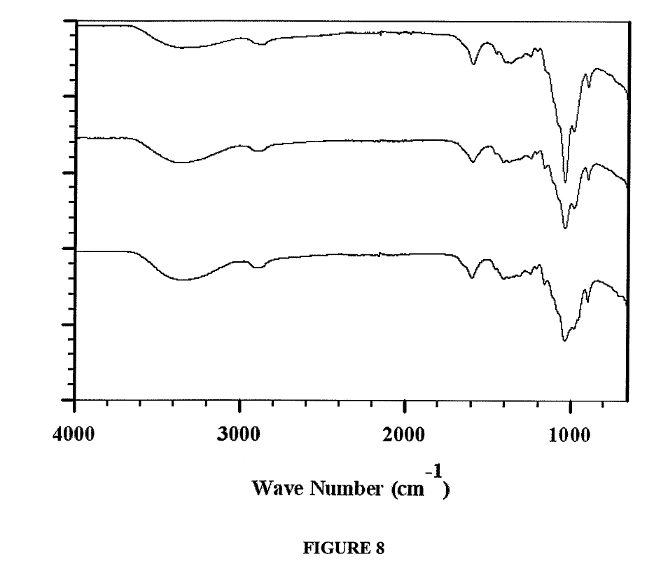

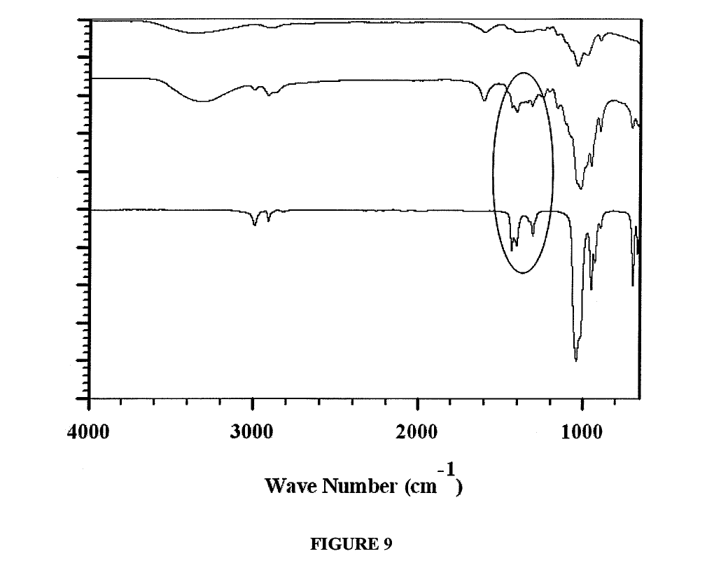

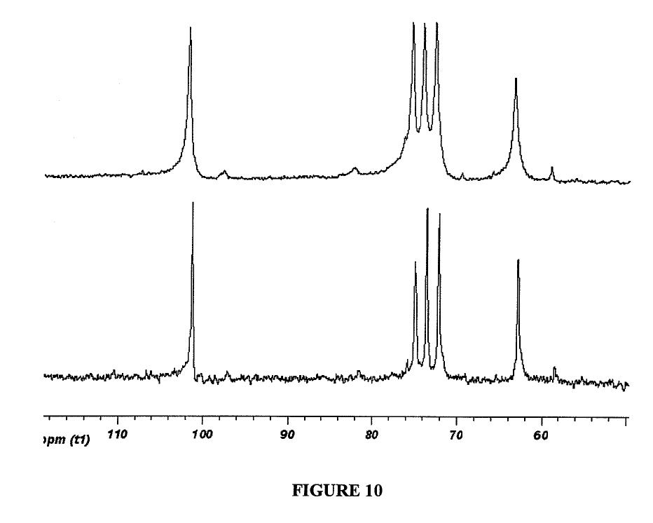

In one aspect, xylan can be removed from a biomass solution using a solvent comprising DMSO. For example, for extraction with DMSO, xylan can be removed at room temperature, or an elevated temperature, e.g., about 50° C. or about 90° C. Using an example method, xylan was removed from a film cast from a biomass solution using both a DMSO solution, and a KOH basic solution, and the xylan was subsequently precipitated out using ethanol, dried, and was characterized by IR (FIGS. 8 and 9) and NMR (FIG. 10). In this example, a total of approximately 0.25 g xylan could be recovered using these methods in both cases.

In one aspect, a KOH solution of water can be used as an extraction solvent for hemicellulose. It will be apparent that the use of this solvent system can be economically viable on a commercial scale.

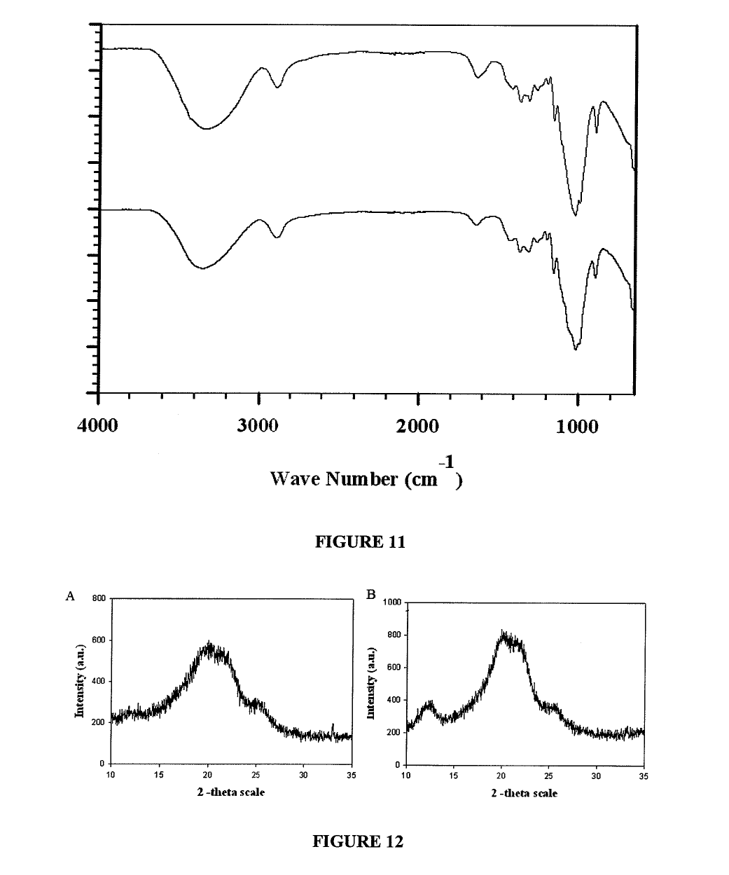

In a further aspect, provided are methods for retrieving cellulose from a biomass solution or film cast therefrom. In one aspect, cellulose removal is carried out after other biomass polymers and/or extractives are removed. Thus, in one aspect, retrieval of cellulose can conveniently be accomplished by first removing other components. Cellulose can remain in a film or biomass solution, in substantially pure form, after the initial separation is carried out. In one example, after xylan extraction, the residual cellulose was collected was found to be colorless and was characterized by IR and XRD analyses. Dry weight of recovered cellulose was found to be approximately 0.32 g. It has been observed before that cellulose films regenerated from plain MCC in IL solution have cellulose II structure. The same was found to be true for this particular cellulose film as well, which was recovered from a combined mixture of biopolymers in IL solution. The cellulose regenerated from solution (without casting a film) was, however, amorphous. Both IR (FIG. 11) and XRD (FIG. 12) analyses of the film confirmed that the structure of cellulose had not been altered due to addition of other biopolymers in the IL-solution.

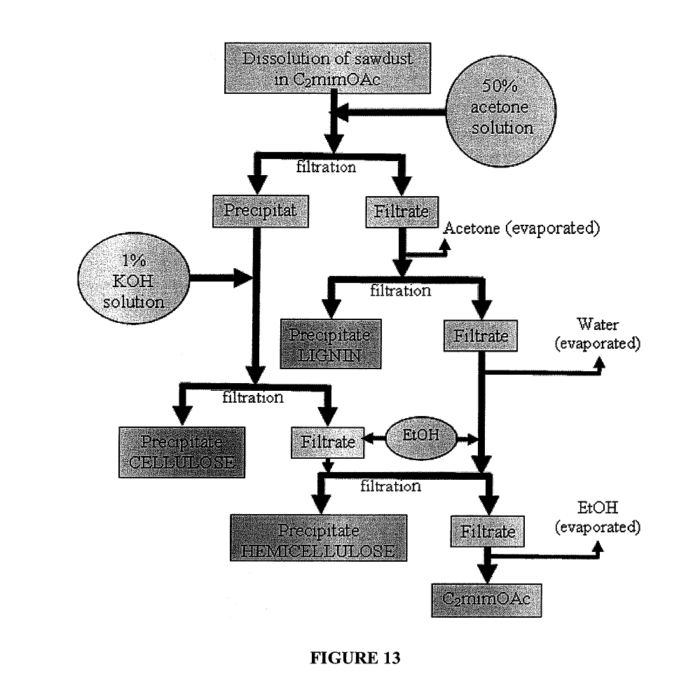

As an example process, FIG. 13 shows a flow that can be used to provide a biomass composition and separate the components therefrom.

Biomass

In the disclosed methods and compositions, biomass is used, fractioned, treated, derivitized, and/or otherwise processed. The term “biomass,” as used herein, refers to living or dead biological material that can be used in one or more of the disclosed processes. Biomass can comprise any cellulosic or lignocellulosic material and includes materials comprising cellulose, and optionally further comprising hemicellulose, lignin, starch, oligosaccharides and/or monosaccharides, biopolymers, natural derivatives of biopolymers, their mixtures, and breakdown products (e.g., metabolites). Biomass can also comprise additional components, such as protein and/or lipid. Biomass can be derived from a single source, or biomass can comprise a mixture derived from more than one source. Some specific examples of biomass include, but are not limited to, bioenergy crops, agricultural residues, municipal solid waste, industrial solid waste, sludge from paper manufacture, yard waste, wood and forestry waste. Additional examples of biomass include, but are not limited to, corn grain, corn cobs, crop residues such as corn husks, corn stover, grasses, wheat, wheat straw, hay, rice straw, switchgrass, waste paper, sugar cane bagasse, sorghum, soy, components obtained from milling of grains, trees (e.g., pine), branches, roots, leaves, wood chips, wood pulp, sawdust, shrubs and bushes, vegetables, fruits, flowers, animal manure, multi-component feed, and crustacean biomass (i.e., chitinous biomass).

Lignocellulosic biomass typically comprises of three major components: cellulose, hemicellulose, and lignin, along with some extractive materials (Sjostorm, E. Wood Chemistry: Fundamentals and Applications, 2nd ed., 1993, New York.). Depending on the source, their relative compositions usually vary to certain extent. Cellulose is the most abundant polymer on Earth and enormous effort has been put into understanding its structure, biosynthesis, function, and degradation (Stick, R. V. Carbohydrates—The Sweet Molecules of Life, 2001, Academic Press, New York.). Cellulose is actually a polysaccharide consisting of linear chain of several hundred to over ten thousand β(1→4) linked D-glucose units. The chains are hydrogen bonded either in parallel or anti-parallel manner which imparts more rigidity to the structure, and a subsequent packaging of bound-chains into microfibrils forms the ultimate building material of the nature.

Hemicellulose is the principal non-cellulosic polysaccharide in lignocellulosic biomass. Hemicellulose is a branched heteropolymer, consisting of different sugar monomers with 500-3000 units. Hemicellulose is usually amorphous and has higher reactivity than the glucose residue because of different ring structures and ring configurations. Lignin is the most complex naturally occurring high-molecular weight polymer (Hon, D. N. S.; Shiraishi, N., Eds., Wood and Cellulosic Chemistry, 2nd ed., 2001, Marcel Dekker, Inc., New York.). Lignin relatively hydrophobic and aromatic in nature, but lacks a defined primary structure. Softwood lignin primarily comprises guaiacyl units, and hardwood lignin comprises both guaiacyl and syringyl units. Cellulose content in both hardwood and softwood is about 43±2%. Typical hemicellulose content in wood is about 28-35 wt %, depending on type of wood. Lignin content in hardwood is about 18-25% while softwood may contain about 25-35% of lignin.

While each of these components could be used in a wide variety of applications including synthesis of platform and commodity chemicals, materials, and production of energy, these components can rarely be separated from biomass in their original form. The principal reason has been the need of a universal processing media for biomass. The components of lignocellulosic biomass are held together by primary lignocellulosic bonds. Lignocellulosic bonds are varied in nature and typically comprise cross-linked networks. Traditionally, lignocellulosic biomass can not be dissolved without degrading in any conventional solvents, and it can be difficult to separate these components in a pure form. However, immense possibilities of separated lignin and hemicellulose-based products have been widely studied (Axegard, P., The Future Pulp Mill—A Biorefinery?, Presentation at 1st International Biorefinery Workshop, Washington, D.C., Jul. 20-21, 2005). The impact of different process options to convert renewable lignocellulosic feedstocks into valuable chemicals and polymers has been summarized by Gallezot (Gallezot, P. Process options for converting renewable feedstocks to bioproducts. Green Chem. 2007, 9, 295-302, which is incorporated by reference herein in its entirety for its teaching of feedstock processing.).

In one aspect, lignocellulosic biopolymers can be separated from a disclosed solution where a few (covalent) up to where substantially all bonds have been broken.

Ionic Liquids

In general, ionic liquids are used to first provide a solution of biomass. It should be appreciated that many in the art have turned to ILs (Rogers and Seddon, Science 2003, 302:792) to solve processing problems due to their non-volatility, solubilizing properties, recycling ability, and ease of processing. ILs can often be viable alternatives to traditional industrial solvents comprising volatile organic compounds (VOCs). In particular, the use of ILs can substantially limit the amount of organic contaminants released into the environment. As such, ILs are at the forefront of a growing field known as “green chemistry.”

Cellulose, an often major component of biomass, for example, has been shown to be capable of dissolution in ILs (Swatloski et al., J Am Chem Soc 2002, 124:4974-4975, PCT Publication No. WO03/029329 A2; Swatloski et al., “Ionic Liquids for the Dissolution and Regeneration of Cellulose,” In Molten Salts XIII: Proceedings of the International Symposium, Trulove, et al., Eds., The Electrochemical Society: Pennington, N.J., 2002, Vol. 2002-19, pp. 155-164, which are incorporated by reference herein for at least their teachings of IL/cellulose dissolution). Components of biomass have also been reportedly dissolved in ILs (WO 05017252; Pu et al., “Ionic liquid as a green solvent for lignin,” J Wood Chem Technol, 2007, 27:23-3, which are incorporated by reference herein in their entireties). It has even been demonstrated that both softwood and hardwood can be directly dissolved in a number of ILs (Fort et al., “Can ionic liquids dissolve wood? Processing and analysis of lignocellulosic materials with 1-n-butyl-3-methylimidazolium chloride,” Green Chem 2007, 9:63-69; Kilpelainen et al., “Dissolution of wood in ionic liquids,” J. Agric. Food Chem. 2007, 55:9142-9148, which are incorporated by reference herein in their entireties). ILs have even been used as a delignification media that allows simultaneous dissolution and delignification of lignocellulosic biomass under microwave heating (see US Application Publication No. 2008/0023162, which is incorporated by reference herein in its entirety). The ionic liquids disclosed in these references can be used in the methods and compositions disclosed herein.

The ionic liquids that can be used in the disclosed methods and compositions comprise ionized species (i.e., cations and anions) and have melting points below about 150° C. For example, the disclosed ionic liquids can be liquid at or below a temperature of about 120° C. or about 100° C., and at or above a temperature of about minus 100° C. or about minus 44° C. For example, N-alkylisoquinolinium and N-alkylquinolinium halide salts have melting points of less than about 150° C. The melting point of N-methylisoquinolinium chloride is 183° C., and N-ethylquinolinium iodide has a melting point of 158° C. In other examples, a contemplated ionic liquid is liquid (molten) at or below a temperature of about 120° C. and above a temperature of about minus 44° C. In some examples, a suitable ionic liquid can be liquid (molten) at a temperature of about minus 10° C. to about 100° C.

Ionic liquids suitable for use herein can be hydrophilic or hydrophobic and can be substantially free of water, a water- or alcohol-miscible organic solvent, or nitrogen-comprising base. Contemplated organic solvents of which the ionic liquid is substantially free include solvents such as dimethyl sulfoxide, dimethyl formamide, acetamide, hexamethyl phosphoramide, water-soluble alcohols, ketones or aldehydes such as ethanol, methanol, 1- or 2-propanol, tert-butanol, acetone, methyl ethyl ketone, acetaldehyde, propionaldehyde, ethylene glycol, propylene glycol, the C₁-C₄ alkyl and alkoxy ethylene glycols and propylene glycols such as 2-methoxyethanol, 2-ethoxyethanol, 2-butoxyethanol, diethyleneglycol, and the like.

Cations

As noted, ionic liquids contain one or more types of cations and one or more types of anions. A suitable cation of a hydrophilic ionic liquid can be cyclic and correspond in structure to a formula shown below:

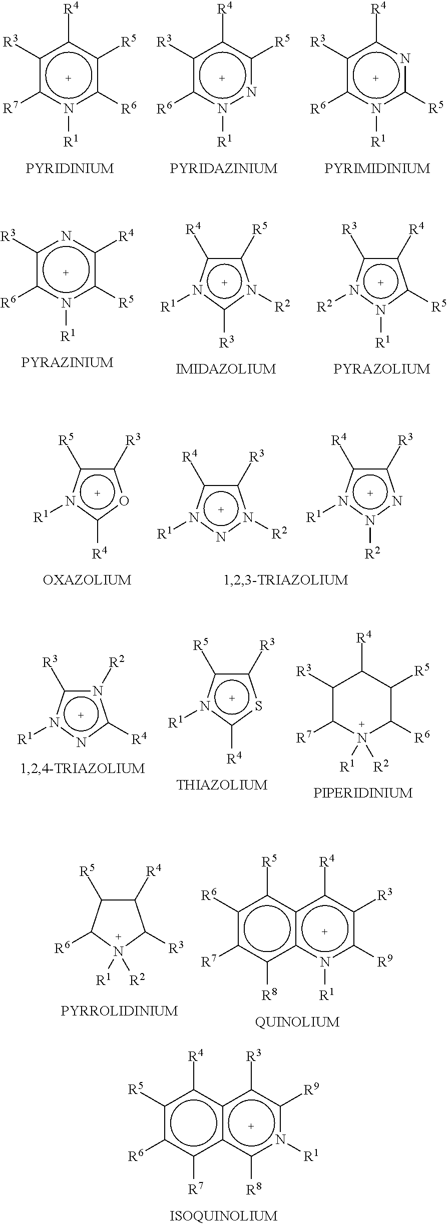

wherein R¹ and R² are independently a C₁-C₆ alkyl group or a C₁-C₆ alkoxyalkyl group, and R³, R⁴, R⁵, R⁶, R⁷, R⁸, and R⁹ (R³-R⁹), when present, are independently H, a C₁-C₆ alkyl, a C₁-C₆ alkoxyalkyl group, or a C₁-C₆ alkoxy group. In other examples, both R¹ and R² groups are C₁-C₄ alkyl, with one being methyl, and R³-R⁹, when present, are H. Exemplary C₁-C₆ alkyl groups and C₁-C₄ alkyl groups include methyl, ethyl, propyl, iso-propyl, butyl, sec-butyl, iso-butyl, pentyl, iso-pentyl, hexyl, 2-ethylbutyl, 2-methylpentyl, and the like. Corresponding C₁-C₆ alkoxy groups comprise the above C₁-C₆ alkyl group bonded to an oxygen atom that is also bonded to the cation ring. An alkoxyalkyl group comprises an ether group bonded to an alkyl group, and here comprises a total of up to six carbon atoms. It is to be noted that there are two isomeric 1,2,3-triazoles. In some examples, all R groups not required for cation formation can be H. Specific examples of such ILs for the dissolution of cellulose are disclosed in U.S. Pat. No. 6,824,599 and Swatloski et al., J Am Chem Soc 2002, 124:4974-4975, which are incorporated by reference herein for there teachings of ionic liquids.

The phrase “when present” is often used herein in regard to substituent R group because not all cations have all of the numbered R groups. All of the contemplated cations comprise at least four R groups, which can, in various examples, be H.

The phrases “substantial absence” and “substantially free” are used synonymously to mean that less than about 5 weight percent, more particularly less than about 1 weight percent, water or other referenced substance is present. For example, it has been reported in Swatloski et al., J Am Chem Soc 2002, 124:4974-4975, that cellulose is no longer soluble in certain ionic liquids if water content is above about 1 weight percent. It should be appreciated, however, that some water may be present since the biomass component is often only partially dry and the ionic liquid itself may contain residual amounts of water. Such residual amounts should be taken into account even though a system is described to be “substantially free of” or “substantially absent” water. The same meaning is intended regarding the presence of a nitrogen-comprising base, alcohol, or otherwise miscible organic solvent.

In one example, all R groups that are not required for cation formation; i.e., those other than R¹ and R² for compounds other than the imidazolium, pyrazolium, and triazolium cations shown above, are H. Thus, the cations shown above can have a structure that corresponds to a structure shown below, wherein R¹ and R² are as described before.

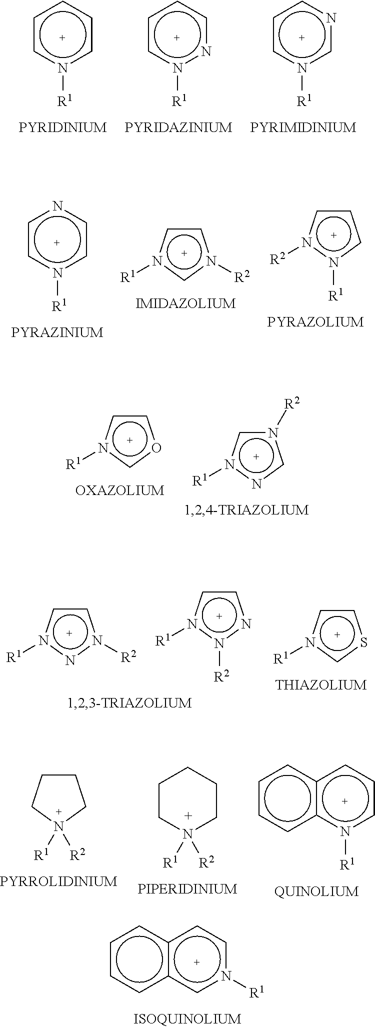

A cation that comprises a single five-membered ring that is free of fusion to other ring structures is also a suitable IL cation for the compositions and methods disclosed herein.

In additional examples, a cation of an ionic liquid can correspond in structure to a formula shown below:

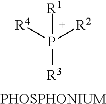

wherein R¹, R², R³, and R⁴, when present, are independently a C₁-C₁₈ alkyl group or a C₁-C₁₈ alkoxyalkyl group.

Still further examples of cations for suitable ILs include ammonium, alkoxyalkyl imidazolium, alkanolyl substituted ammonium, alkoxyalkyl substituted ammonium, aminoalkyl substituted ammonium.

Anions

An anion for a contemplated ionic liquid cation can be a halide (fluoride, chloride, bromide, or iodide), perchlorate, a pseudohalide, or C₁-C₆ carboxylate. Pseudohalides are monovalent and have properties similar to those of halides (Schriver et al., Inorganic Chemistry, W. H. Freeman & Co., New York, 1990, 406-407). Pseudohalides include the cyanide (CN⁻), thiocyanate (SCN⁻), cyanate (OCN⁻), fulminate (CNO⁻), azide (N₃⁻), tetrafluoroborate (BF₄), and hexafluorophosphate (PF₆)anions.

Carboxylate anions that comprise 1-6 carbon atoms (C₁-C₆ carboxylate) are illustrated by formate, acetate, propionate, butyrate, hexanoate, maleate, fumarate, oxalate, lactate, pyruvate, and the like, are also suitable for appropriate contemplated ionic liquid cations. Further examples include sulfonated or halogenated carboxylates.

Sulfate anions, such as tosylate, mesylate, trifluoromethanesulfonate, trifluoroethane sulfonate, di-trifluoromethanesulfonyl amino, docusate, and xylenesulfonate (see WO2005017252, which is incorporated by reference herein for ionic liquids with anions derived from sulfonated aryls) are also suitable for use as the anionic component of an IL.

Still other examples of anions that can be present in the disclosed ILs include, but are not limited to, other sulfates, sulfites, phosphates, phosphonates (see Fukaya et al., Green Chem, 2008, 10:44-46), phosphites, nitrate, nitrites, hypochlorite, chlorite, perchlorate, bicarbonates, and the like, including mixtures thereof.

### SPECIFIC EXAMPLES

Suitable ILs for the disclosed compositions and methods can comprise any of the cations and anions disclosed herein. For example, a suitable ionic liquid can be 1-alkyl-3-methylimidazolium halide, 1-alkyl-3-methylimidazolium C₁₋₆ carboxylate. Some specific examples of suitable ILs that can be used in the disclosed compositions and methods include, but are not limited to, allylmethylimidazolium Cl, allylbutylimidazolium Cl, diallylimidazolium Cl, allyloxymethylimidazolium Cl, allylhydroxyethylimidazolium Cl, allylmethylimidazolium formate, allylmethylimidazolium OAc, benzylmethylimidazolium Cl, bis(methylimidazolium)sulfoxide Cl, ethylmethylimidazolium benzoate, ethylmethylimidazolium CF₃SO₃, ethylmethylimidazolium Cl, ethylmethylimidazolium OAc, ethylmethylimidazolium xylenesulfonate, ethylmethylimidazolium methylphosphonate, propylmethylimidazolium formate, butylmethylimidazolium BF₄, butylmethylimidazolium Cl, butylmethylimidazolium Cl+FeCl₃, butylmethylimidazolium MeSO₄, butylmethylimidazolium (CN₂)N—, butylmethylimidazolium PF₆, butyl-2,3-dimethylimidazolium Cl, methylhydroxyethylimidazolium Cl, N,N′-dimethylimidazolium Cl, N,N′-dimethylimidazolium MeSO₄, N,N′-dimethylimidazolium OAc, 1-(2-hydroxylethyl)-3-methylimidazoium Cl, 1-methyl-3-(4-vinylbenzyl)imidazolium Cl, 3,3-ethane-1,2-dylbis(methylimidazolium) dichloride, 3,3-ethane-1,2-dylbis(methylimidazolium) dichloroaluminate, 1-vinyl-3-(4-vinylbenzypimidazolium Cl, diethyl N-methyl-N-(2-methoxyethyl)ammonium Tf₂N, hydroxybutyl trimethylammonium carbamate, nitronium Tf₂N, tetrabutylammonium benzoate, tetrabutylammonium, dodecylbenzenesulfonate, tetrabutylammonium OH, tetrabutylammonium xylenesulfonate, phenyltributylammonium xylenesulfonate, allylmethylpyridinium Cl, benzylpyridinium Cl, butylmethyl pyrrolidinium 4-hydroxybenzenesulfonate, ethylpyridinium Br, trihexyltetradecylphosphonium xylenesulfonate, choline Cl+urea, choline Cl+ZnCl₂.

Some additional examples of ionic liquids include, but are not limited to, the following quaternary ammonium salts: Bu₄NOH, Bu₄N(H₂PO₄), Me₄NOH, Me₄NCl, Et₄NPF₆, and Et₄NCl.

In various examples disclosed herein, biomass, optionally including cellulose and other biopolymers, can be partially or completely dissolved with or without derivatization in the disclosed fractionation compositions comprising ionic liquids and fractionation polymer. A contemplated solution of biomass in the ionic liquid portion of the fractionation composition can contain cellulose in an amount of from about 5 to about 35 wt. %, from about 5 to about 25 wt. %, from about 5 to about 20 wt. %, from about 5 to about 15 wt. %, from about 10 to about 35 wt. %, from about 10 to about 25 wt. %, from about 15 to about 35 wt. %, or from about 15 to about 25 wt. % of the solution. In other examples, the ionic liquid can contain cellulose in an amount of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 wt. % of the solution, where any of the stated values can form an upper or lower endpoint. Further, a solution of biomass in an ionic liquid can contain cellulose in an amount of from about 5 to about 35 parts by weight, from about 5 to about 25 parts by weight, from about 5 to about 20 parts by weight, from about 5 to about 15 parts by weight, from about 10 to about 35 parts by weight, from about 10 to about 25 parts by weight, from about 15 to about 35 parts by weight, or from about 15 to about 25 parts by weight of the solution. In other examples, the ionic liquid can contain cellulose in an amount of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 parts by weight of the solution, where any of the stated values can form an upper or lower endpoint.

The disclosed fractionation compositions and methods can also comprise mixtures of two, or more, ILs in any suitable combination. In certain examples, one can use one IL that is selective for cellulose and another IL (miscible or immiscible with the first) that is selective for lignin.

Processing of Biomass in ILs

Since ILs can dissolve major components of biomass (e.g., cellulose, lignin, and hemicellulose) without any pretreatment, ILs with dissolved/suspended processing aids or other additives, can allow simultaneous dissolution and processing of biomass. As such, in many examples herein, disclosed are methods and compositions that involve the processing of biomass (or its components) in one or more ILs and with one or more processing aids that are simultaneously dissolved (or suspended) in the IL.

In a specific example of this embodiment, a biomass (e.g., lignocellulosic, crustacean, or other type of biomass) is completely or partially dissolved or suspended in an ILs at up to 50 wt %. A processing aid can already be present in the IL or can be added after the biomass is dissolved. The catalysts and any optional additives can be used to increase dissolution, facilitate disintegration, cleave bonds, separate biopolymers from biomass, and for derivatization and other treatments of biomass and their components.

Depending on the processing aids, the mixture can be heated up to about 250° C., or 150° C. Such heating can involve microwave, infrared, or ultrasound irradiation, and/or other external sources of energy supply. Heating can be performed for up to 16 hours or longer. Reactions can be held in air or under inert environment depending on catalyst(s) and additive(s) used.

All the components of biomass can be dissolved simultaneously (or selectively) and optionally regenerated separately later using appropriate regeneration solvents. Likewise, the processing aids can be recovered from the solution and re-used.

Processing Aids

Processing aids can be added to the system in order to stiochiometrically/nonstoichiometrically interact with biomass or their biopolymer components to increase dissolution, facilitate disintegration, cleave bonds, delignifying, fermentate, separate biopolymers from biomass, and for derivatization and other treatments of biomass and their components. Any processing aid can be used in these methods as long as the ionic liquid media does not inactivate the processing aid. Suitable processing aids are those that can selectively cleave lignin from lignocellulosic biomass or degrade a biopolymer component of biomass (e.g., fermentation of sugars into ethanol). Some specific examples of processing aids, include but are not limited to, catalysts, metal salts, polyoxymetalates (POMs) (e.g., H₅[PV₂Mo₁₀O₄₀]), anthraquinone, enzymes, and the like. Dichloro dicyano quinone (DDQ) is an example of one type of processing aid that can selectively cleave lignocellulosic bonds in solution and help separating components of lignocellulosic biomass. In many examples, the processing aid is not an acid catalyst.

Also, contemplated herein are processing aids like microwave or thermal irradiation. Such aids can likewise be used to break bonds in a biomass material present in an IL.

Multiple-IL Systems

In a still further embodiment, a mixture of two or more different ILs can be used as media for processing biomass and its components. That is, ILs with specific properties can be mixed together to yield a media with desired properties required for processing a wide variety of biomass materials. For example, one can use a first IL that is selective for lignin to delignify a lignocellulosic biomass, whereas another IL (whether miscible or immiscible with the first IL) can be used to dissolve cellulose. Both ILs can be present in the multiple-IL system. Such multi-IL systems can be used directly for processing biomass or, alternatively, they can be combined with a fractionation polymer in order to fraction certain components in the biomass, as disclosed above.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, pH, etc.) but some errors and deviations should be accounted for. Unless indicated otherwise, parts are parts by weight, temperature is in ° C. or is at ambient temperature, and pressure is at or near atmospheric. There are numerous variations and combinations of conditions, e.g., component concentrations, temperatures, pressures, and other reaction ranges and conditions that can be used to optimize the product purity and yield obtained from the described process. Only reasonable and routine experimentation will be required to optimize such process conditions.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Microcrystalline cellulose (MCC) and xylan from beechwood (both from Sigma-Aldrich) were studied as cellulose and hemicellulose standards, respectively. Indulin AT (from MeadWestvaco), which is known to be the purified form of pine lignin obtained from Kraft process (completely free of hemicellulosic materials), was studied as the lignin standard. ILs 1-ethyl-3-methylimidazolium chloride (C₂mimCl), 1-butyl-3-methylimidazolium chloride (C₄mimCl), and 1-ethyl-3-methylimidazolium acetate (C₂mimOAc) were provided by BASF (Florham Park, N.J.) and were used as received. Other reagents (mentioned later in Table 2) were obtained from Sigma-Aldrich and were used without any further treatment. Deionized water was used throughout the experiments which was obtained from a commercial deionizer (Culligan, Northbrook, Ill.) and had specific resistivity of 17.25 MΩ-cm at 25° C.

### Example 1

### Solubility Measurement

Ten grams of ILs were taken in different vials and the lignocellulosic standards were added in small increments (about 0.5 g) and dissolved by heating in an oven at about 90° C. with occasional vortexing and/or mechanical stirring. Further additions of standards were stopped as insoluble particles were observed or the solutions became too viscous to be stirred mechanically. Solubilities of these standards in regeneration solvents were determined in similar way at room temperature (about 23° C.) by adding the standards at about 0.1 g increments. Only, solubilities of the standards in DMSO were studied at elevated temperature, just like in the ILs. Since, these conventional solvents possess much less viscosity compared to the ILs, increased viscosity of solution with addition of biopolymer standards was never an issue. Further additions of standards were stopped as the added particles were found to settle at the bottom after centrifugation indicating proximity to saturation point.

### Example 2

### Solution-Based Separation

C₂mimOAc was chosen for subsequent studies since it is liquid at room temperature and could be studied at lower temperatures without worrying about crystallization/solidification of the IL. 0.33 g of each of the standards were mixed together and then dissolved in 10 g of C₂mimOAc at about 90° C. The viscous solution then washed thoroughly with 100 mL of water so that water washes up all the IL from the solution. The remaining agglomerated chunk of biopolymers was washed with 30 mL of 1:1 acetone-water solution to extract Indulin AT. The undissolved materials, separated by centrifuging, were washed with 50 g of 1 wt % KOH aqueous solution. This was done to dissolve xylan present in the mixture. Finally, the undissolved materials in KOH solution was separated by centrifuging, washed with water and dried in oven at about 85° C.

### Example 3

### Film-Based Separation

In another run, the biopolymer standards dissolved in IL was cast as a film on a glass plate and the plate was immersed completely in water at room temperature. C₂mimOAc was thus washed out. The film had a brownish appearance mainly because of Indulin AT. The film was then soaked in 1:1 acetone-water solution to extract Indulin AT. In order to remove xylan from the film, the film was cut into two halves which were treated with 1 wt % aqueous KOH solution at room temperature and with DMSO at about 50° C., respectively. Residual cellulose was recovered as a transparent film and was rinsed with water.

In both Example 1 and Example 2, C₂mimOAc was recovered by evaporating water. Indulin AT was recovered from 1:1 acetone-water solution by evaporating acetone and separating precipitated Indulin AT from water by centrifugation. Xylan was precipitated from KOH solution by adding ethanol, and later separated by centrifugation. All the recovered materials were dried in oven at about 85° C. Original and regenerated samples were characterized by NMR, IR, and PXRD.

Samples from Example 1 and Example 2 were characterized. NMR studies were performed using a Bruker AVANCE 500 NMR spectrometer with a 5 mm BBO probe. Cellulose samples (original and regenerated) were dissolved in C₂mimOAc/DMSO-d6 (85:15) and ¹³C NMR spectra were collected at 70° C. A total of 20,000 scans were collected for ¹³C NMR at 125.76 MHz and spectra were processed with a 10 Hz line-broadening factor. Lignin and hemicellulose (original and regenerated) were dissolved directly in DMSO-d6 and ¹³C NMR spectra were collected at room temperature with 5000 scans. All the ¹H NMR spectra were collected with 128 scans at 500.13 MHz. IR spectra were taken by a PerkinElmer Spectrum 100 FT-IR spectrometer using 4 scans with a resolution of 4 cm⁻¹. PXRD spectra were collected using a Philips APD 3830 powder X-ray diffractometer with Cu source/tube and graphite monochromator. Scan speed was 0.1° (2θ)/sec and time per step was 0.2 sec.

### SPECIFIC EMBODIMENTS

In one aspect, a biomass extraction process comprises providing a composition comprising a biomass substantially dissolved in an ionic liquid, the biomass comprising cellulose, lignin, and at least one xylan, wherein the composition and/or the ionic liquid is substantially free of water; and separating at least a portion of the at least one xylan from the composition. The biomass can be a lignocellulosic biomass. The biomass can also be derived from a natural source, such as, for example, softwood, hardwood, or a combination thereof.

In a further aspect, providing the composition comprises dissolving the biomass in the ionic liquid at a temperature of from about 0° C. to about 250° C., of from about 0° C. to about 100° C., from about 40° C. to about 100° C.

In one aspect, the biomass is substantially separated from the ionic liquid prior to separating at least a portion of the at least one xylan from the composition. In a further aspect, a biomass non-solvent is added to the composition in an amount effective to substantially precipitate the biomass from the ionic liquid, thereby forming a precipitated biomass.

In one aspect, at least a portion of the at least one xylan is separated from the precipitated biomass using an aqueous basic solution, dimethyl sulfoxide, or a combination thereof In a further aspect, at least a portion of the lignin is separated from the precipitated biomass using a lignin solvent. In one aspect, at least a portion of the lignin solvent is an acetone/water mixture.

In one aspect, a biomass film is formed from the composition, and the ionic liquid is removed from the film. In a further aspect, a method further comprises separating at least a portion of the lignin from the composition prior to separating at least a portion of the at least one xylan from the composition. In yet a further aspect, at least a portion of the lignin is separated from the composition using a water/acetone mixture.

In one aspect, a method further comprises separating at least a portion of the cellulose from the composition. In a further aspect, a method further comprises separating at least a portion of the ionic liquid from the composition.

In one aspect, the ionic liquid comprises one or more cations and one or more anions and wherein the cations comprise one or more compounds having the formula

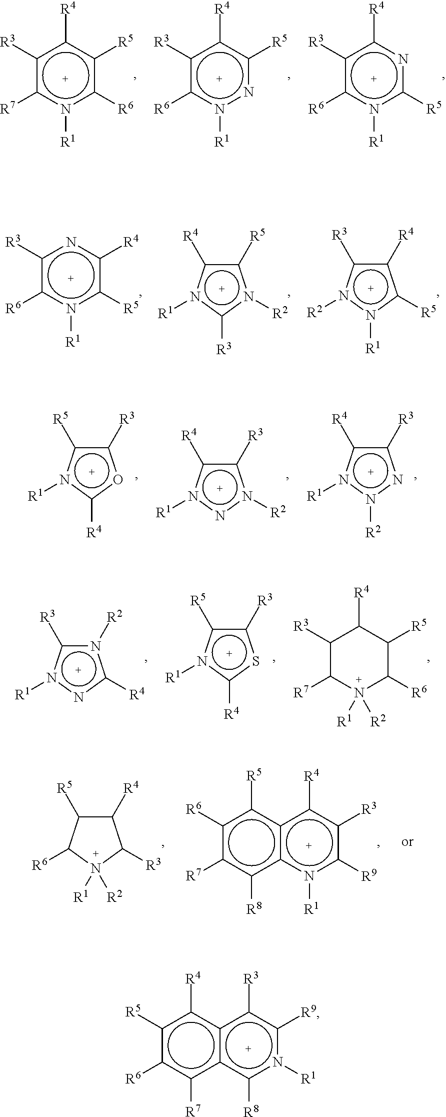

wherein R¹ and R² are independently a C₁-C₆ alkyl group or a C₁-C₆ alkoxyalkyl group, and R³, R⁴, R⁵, R⁶, R⁷, R⁸, and R⁹ are independently H, a C₁-C₆ alkyl, a C₁-C₆ alkoxyalkyl group, or a C₁-C₆ alkoxy group, and the anions comprise F, Cl⁻, Br⁻, I⁻, ClO₄⁻, BF⁴⁻, PF₆⁻, AsF₆⁻, SbF₆, NO₂⁻, NO₃⁻, SO₄²⁻, PO₄³⁻, HPO₄²⁻, CF₃CO₂⁻, CO₃²⁻, or C₁-C₆ carboxylate.

In a further aspect the ionic liquid comprises one or more cations and one or more anions and wherein the one or more cations comprise one or more compounds having the formula:

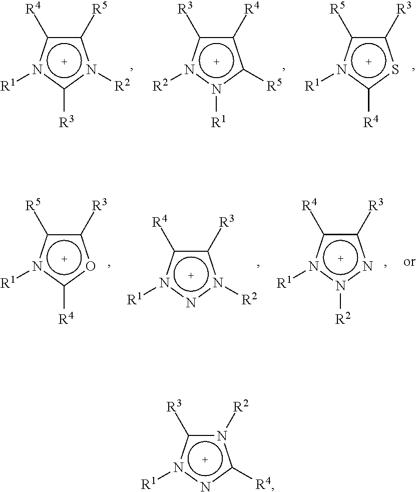

wherein R¹ and R² are independently a C₁-C₆ alkyl group or a C₁-C₆ alkoxyalkyl group, and R³, R⁴, and R⁵ are independently H, a C₁-C₆ alkyl group, a C₁-C₆ alkoxyalkyl group, or a C₁-C₆ alkoxy group, and the anions comprise one or more of F⁻, Cl⁻, Br⁻, I⁻, ClO₄⁻, BF₄⁻, PF₆⁻, AsF₆⁻, SbF₆, NO₂⁻, NO₃⁻, SO₄²⁻, PO₄³⁻, HPO₄²⁻, CF₃CO₂⁻, CO₃²⁻, or C₁-C₆ carboxylate.

In one aspect, the one or more cations comprise an imidazolium ion having the formula:

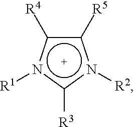

wherein R¹ and R² are C₁-C₆ alkyl. In a further aspect, R¹ or R² is methyl.

In one aspect, R¹ is C₁-C₄-alkyl and R² is methyl. In a further aspect, R³, R⁴, and R⁵ each are H. In yet a further aspect, the ionic liquid comprises 1-(C₁-C₆ alkyl)-3-methyl-imidazolium halide. In a still further aspect, the ionic liquid comprises 1-(C₁-C₆ alkyl)-3-methyl-imidazolium C₁-C₆ carboxylate.

In one aspect, the at least one xylan is a component of a hemicellulose that is present in the composition. In a further aspect, the at least one xylan is xylan, glucoronoxylan, arabinoxylan, or a combination thereof.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A biomass extraction process comprising:
(a) providing a composition comprising a biomass substantially dissolved in an ionic liquid, the biomass comprising cellulose, lignin, and at least one xylan,
wherein the ionic liquid is substantially free of water; and
(b) separating at least a portion of the at least one xylan from the composition.

2. The process of claim 1, wherein the biomass is a lignocellulosic biomass.

3. The process of claim 1, wherein the biomass is derived from a natural source.

4. The process of claim 1, wherein the biomass is derived from softwood, hardwood, or a combination thereof.

5. The process of claim 1, wherein providing the composition comprises dissolving the biomass in the ionic liquid at a temperature of from about 0° C. to about 250° C.

6. The process of claim 1, wherein providing the composition comprises dissolving the biomass in the ionic liquid at a temperature of from about 0° C. to about 100° C.

7. The process of claim 1, wherein providing the composition comprises dissolving the biomass in the ionic liquid at a temperature of from about 40° C. to about 100° C.

8. The process of claim 1, wherein the biomass is substantially separated from the ionic liquid prior to separating at least a portion of the at least one xylan from the composition.

9. The process of claim 8, wherein a biomass non-solvent is added to the composition in an amount effective to substantially precipitate the biomass from the ionic liquid, thereby forming a precipitated biomass.

10. The process of claim 9, wherein at least a portion of the at least one xylan is separated from the precipitated biomass using an aqueous basic solution, dimethyl sulfoxide, or a combination thereof.

11. The process of claim 9, wherein at least a portion of the lignin is separated from the precipitated biomass using a lignin solvent.

12. The process of claim 10, wherein at least a portion of the lignin solvent is an acetone/water mixture.

13. The process of claim 8, wherein a biomass film is formed from the composition, and the ionic liquid is removed from the film.

14. The process of claim 1, further comprising separating at least a portion of the lignin from the composition prior to separating at least a portion of the at least one xylan from the composition.

15. The process of claim 14, wherein at least a portion of the lignin is separated from the composition using a water/acetone mixture.

16. The process of claim 1, further comprising separating at least a portion of the cellulose from the composition.

17. The process of claim 1, further comprising separating at least a portion of the ionic liquid from the composition.

18. The process of claim 1, wherein the ionic liquid comprises one or more cations and one or more anions and wherein the cations comprise one or more compounds having the formula 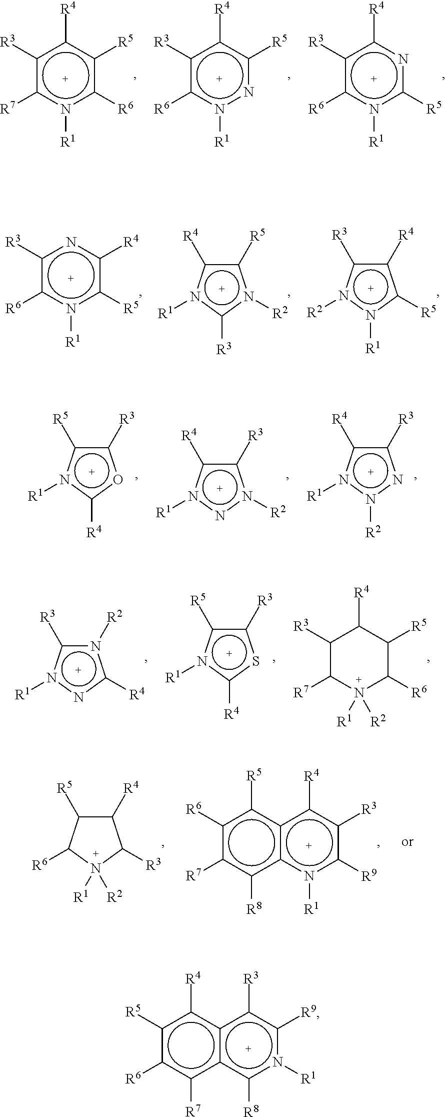
wherein R¹ and R² are independently a C₁-C₆ alkyl group or a C₁-C₆ alkoxyalkyl group, and R³, R⁴, R⁵, R⁶, R⁷, R⁸, and R⁹ are independently H, a C₁-C₆ alkyl, a C₁-C₆ alkoxyalkyl group, or a C₁-C₆ alkoxy group, and the anions comprise F⁻, Cl⁻, Br⁻, I⁻, ClO₄⁻, BF₄⁻, PF₆⁻, AsF₆⁻, SbF₆, NO₂⁻, NO₃⁻, SO₄²⁻, PO₄³⁻, HPO₄²⁻, CF₃CO₂⁻, CO₃²⁻, or C₁-C₆ carboxylate.

19. The method of claim 1, wherein the ionic liquid comprises one or more cations and one or more anions and wherein the one or more cations comprise one or more compounds having the formula: 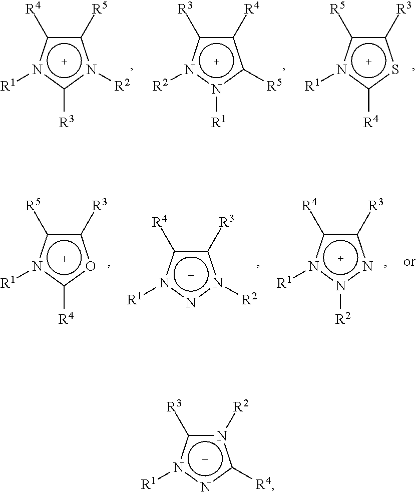
wherein R¹ and R² are independently a C₁-C₆ alkyl group or a C₁-C₆ alkoxyalkyl group, and R³, R⁴, and R⁵ are independently H, a C₁-C₆ alkyl group, a C₁-C₆ alkoxyalkyl group, or a C₁-C₆ alkoxy group, and the anions comprise one or more of F⁻, Cl⁻, Br⁻, I⁻, ClO₄⁻, BF₄⁻, PF₆⁻, AsF₆⁻, SbF₆, NO₂⁻, NO₃⁻, SO₄²⁻, PO₄³⁻, HPO₄²⁻, CF₃CO₂⁻, CO₃²⁻, or C₁-C₆ carboxylate.

20. The process of claim 18, wherein the one or more cations comprise an imidazolium ion having the formula: 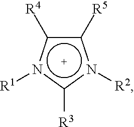
wherein R¹ and R² are C₁-C₆ alkyl.

21. The process of claim 20, wherein R¹ or R² is methyl.

22. The process of claim 20, wherein R¹ is C₁-C₄-alkyl and R² is methyl.

23. The process of claim 20, wherein R³, R⁴, and R⁵ each are H.

24. The process of claim 20, wherein the ionic liquid comprises 1-(C₁-C₆ alkyl)-3-methyl-imidazolium halide.

25. The process of claim 20, wherein the ionic liquid comprises 1-(C₁-C₆ alkyl)-3-methyl-imidazolium C₁-C₆ carboxylate.

26. The process of claim 1, wherein the at least one xylan is a component of a hemicellulose that is present in the composition.

27. The process of claim 1, wherein the at least one xylan is xylan, glucoronoxylan, arabinoxylan, or a combination thereof.
